(19) United States
(12) Patent Application Publication
(10) Pub. No.: US 2023/0065690 A1
(43) Pub. Date: Mar. 2, 2023

(54) Soil Penetrating Probe and System for Measuring Electrical Properties to Determine Soil Water Content

(71) Applicant: Landscan, LLC, Lake Oswego, OR (US)

(72) Inventors: Stephen P. Farrington, Gaysville, VT (US); Rocky Hull, South Royalton, VT (US); Daniel James Rooney, Lake Oswego, OR (US); Gregory Anderson, San Diego, CA (US)

(73) Assignee: Landscan, LLC, Lake Oswego, OR (US)

(21) Appl. No.: 17/411,476

(22) Filed: Aug. 25, 2021

(51) Int. Cl. G01N 27/22; G01N 33/24

(52) U.S. Cl. CPC G01N 27/223; G01N 27/221; G01N 33/246

(57) ABSTRACT

A probe for penetrating and measuring electrical properties of a soil comprises a probe tip connected, via a coaxial cable, to electrical circuitry. The probe tip is convex and includes first and second electrodes with an electrode insulator therebetween. The first electrode is tubular and includes an interior surface defining a central opening extending through the first electrode. The second electrode includes a convex section extending away from the first electrode, and the convex section is configured for insertion into soil. The one end of the coaxial cable is disposed within the central opening of the first electrode, and the inner conductive core of one end of the coaxial cable connected to the second electrode, and the conductive shield of the one end of the coaxial cable connected to the first electrode.

### BACKGROUND OF THE INVENTION

### Field of the Invention

The present invention relates to the field of in-situ sensing of soil and other media constituents. More specifically, it relates to a penetrometer or probe that may be advanced into soil while simultaneously measuring, with minimal soil disturbance, the electrical impedance of the soil and determining the soil-water content.

### 2. BRIEF DESCRIPTION OF THE RELATED ART

Cone penetrometers are often equipped with electrodes that are utilized to measure soil electrical properties. Electrodes are positioned on the penetrometer such that each electrode may have direct contact with the soil. Most conventional configurations of the placement of the electrodes on the penetrometer fall into two broad categories of physical configuration that each include distinct limitations and disadvantages.

In one of the conventional physical configurations, an example of which is shown in U.S. Pat. No. 1,910,021 issued to Legg, the electrodes are cylindrical or frustoconical in shape, forming rings around the outer surface of the cylindrical body of the penetrometer probe. Between the electrodes, non-metallic parts are required to insulate the electrodes from one another. Due to their relatively weak material composition, the electrodes and non-metallic parts are principally non-load bearing. Therefore, the axial load on the probe, due to penetration, must be carried by an interior strength member of smaller cross-sectional area and moment of inertia than the probe body. As a result, the probe is weakened in the vicinity of the electrodes, and there is a reduction in the available space, within the probe, to house other sensor modules and wiring.

In the second prior physical configuration, as shown for example in U.S. Pat. No. 5,497,091, disk-shaped conductive electrodes are disposed in an electrically insulating element or strip that is disposed in the cylindrical outer surface of the probe such that the discs are side facing and aligned with each other vertically. Also, this second configuration has the disadvantage that the electrical properties measured are representative of soil contacting only a small fraction of the perimeter of the probe, corresponding to a small angular range about the vertical axis of the probe. This is a disadvantage when correlating the electrical properties measurements made using this arrangement of electrodes to measurements from additional probe sensors such as tip stress and sleeve friction which interrogate a volume of soil centered about the longitudinal axis of the probe.

Several other disadvantages are common to both configurations. The electrical field resulting from potential or current applied to the electrodes is predominantly contained within the bounding volume of each probe, and the amount of fringing field actually in the soil represents only a small fraction of the overall spatial distribution of the electric field intensity. Thus, the probes have limited sensitivity to contrasts in soil electrical properties.

Second, the probes are only able to measure in situ electrical properties of soils which have already been profoundly disturbed by the penetration process. As the probes move through the soil, the electrodes significantly trail the conical tip. Well before the soil electrical properties are measured, the soil has been radially displaced by the probe and compacted into surrounding soils. The soil grains have been profoundly re-arranged, and the porosity reduced. As a result, the volumetric soil water content has been changed by forceful passage of the penetrometer through the soil prior to the water content being measured. The soil electrical conductivity has likewise been changed by compressing the charge-carrying surface area of the soil grains into a smaller volume.

Third, the depth at which the soil electrical properties are measured by the aforementioned probe configurations is a disadvantage. Since the electrodes are a distance from the conical tip, the soil electrical properties are measured at a different depth from the depth of soil acting on either the probe tip or friction sleeve. As a result, depth correction calculations are required to align measurements with respect to depth, and water content measurements are never obtained from as great a soil depth as are tip stress, sleeve friction, and other measurements whose sensors are positioned closer to the distal end of the probe than are the electrodes. If penetration depth is limited by the length of the probe or by encountering too much resistance to penetrate, then the soil data profile will be lacking water content information where other sensor information is available.

Additional configurations of penetrometers attempt to overcome some of the disadvantages, discussed above, of the cylindrical and disc electrode configurations. For example, US Patent Application 2010/0257920A includes a penetrometer including a conical tip electrode connected to a cylindrical electrode. The conical tip electrode forms the point of entry into the soil and is the first part of the penetrometer to touch newly encountered soil as the depth of penetration testing increases.

Even though the electrodes are formed as part of the penetrometer casing, this configuration also has disadvantages. First, the type of wiring and circuitry utilized has the potential for signal interference with any other types of sensors which may be located in the penetrometer. For example, there may be other sources of electromagnetism within the probe such as additional sensors and the reabsorption of EM emissions radiated from the signal path of the electrical properties sensor itself and reflected off the inside of the probe. This interference negatively effects the accuracy of electrical properties measurements made using the conical tip electrode as well as the accuracy of any other sensors located in the penetrometer.

Second, the physical configuration of the conical tip electrode, cylindrical electrode and wiring utilizes valuable space in and on the penetrometer. For ease of insertion into the soil, penetrometers must be formed with relatively narrow diameters or widths. As a result, penetrometers have a very limited amount space in which to position multiple sensors. The multiple wires connected to the electrodes as well as the vertical length of the conical and cylindrical electrodes is utilizing space and preventing additional sensors from being added.

Accordingly, there is a need for an improved penetrometer capable of making more accurate measurements of the electrical properties and other sensed properties of soil, before the soil is disturbed, while also providing space for additional sensors.

### BRIEF SUMMARY OF THE INVENTION

One embodiment of the invention, according to this disclosure is a probe for penetrating and measuring electrical properties of a soil. The probe including a tubular first electrode including an interior surface defining a central opening extending through the first electrode; a second electrode including a convex section extending away from the first electrode, the convex section configured for insertion into soil; an electrode insulator between the first and second electrodes; and a coaxial cable within the central opening of the first electrode, the coaxial cable including an inner conductive core surrounded by a conductive shield with a core insulator therebetween, the inner conductive core of one end of the coaxial cable connected to the second electrode, and the conductive shield of the one end of the coaxial cable connected to the first electrode.

In another aspect of the embodiment, the probe includes a sensor body connected to the first electrode; and a probe tip insulator disposed between the sensor body and the first electrode, the probe tip insulator extending substantially radially.

In another aspect of the embodiment, the probe includes an electrical circuit connected to a second end of the coaxial cable, the electrical circuit configured to transmit, via the coaxial cable, electrical signals to the first and second electrodes. The electrical circuit may also comprise a Clapp oscillator.

In another aspect of the embodiment, the probe includes a probe housing connected to the second electrode and the coaxial cable extending from the first and second electrode through the probe housing; and a distal end of the coaxial cable extending outside the probe housing, the distal end of the coaxial cable connected to the electrical circuit outside the probe housing.

In another aspect of the embodiment, the tubular first electrode includes a first end surface and a second end surface with the interior surface extending within the tubular electrode from the first end surface to the second end surface, and the central opening is defined by the interior surface extending through the first electrode, first end surface and the second end surface.

In another aspect of the embodiment, the second electrode includes a third end surface opposite the convex portion; and a tip stem portion on the third end surface, the tip stem portion extending away from the convex portion, the tip stem portion having a smaller width than the third end surface and the tip stem portion positioned within the central opening of the first electrode.

In another aspect of the embodiment, the electrode insulator extends into the central opening of the first electrode and the electrode insulator is between the tip stem portion of the second electrode and the interior surface of the first electrode.

In another aspect of the embodiment, the probe includes a cavity within the tip stem portion of the second electrode, the cavity configured to receive the conductive core of the coaxial cable.

In another aspect of the embodiment, the convex portion is solid.

In another aspect of the embodiment, the convex portion includes a conical exterior surface.

In another aspect of the embodiment, the probe includes a conductive member disposed on the electrode insulator within the central opening, and the conductive member connected to the first electrode and the conductive shield of the coaxial cable.

In another aspect of the embodiment, the conductive member is an annular disc with a hollow center, and the inner conductive core and cable insulator are within the hollow center.

In another aspect of the embodiment, the first electrode, second electrode and insulator are concentrically connected.

In another aspect of the embodiment, the first electrode and second electrode are comprised of a metal or a metallic compound.

In another aspect of the embodiment, the first electrode comprises a first compound and the second electrode comprises a second compound, wherein the first compound and second compound comprise different metals or metallic compounds.

A second embodiment of the invention, according to this disclosure, is a method of making a probe to measure electrical properties of soil or medium. The method includes the steps of:

providing a tubular first electrode including an interior surface defining a first electrode central opening extending through first electrode;

providing a second electrode including a convex portion configured for insertion into the soil;

providing an electrode insulator configured to be placed between the first and second electrodes and inside the first electrode central opening;

inserting the electrode insulator into to the central opening of the first electrode such that the electrode insulator and first electrode are connected;

connecting the second electrode to the electrode insulator;

providing a coaxial cable with one end including an inner conductive core, a conductive shield, and a core insulator therebetween;

inserting the one end of the coaxial cable through the first electrode central opening;

connecting the inner conductive core of the one end of coaxial cable to the second electrode such that the inner conductive core and the second electrode are electrically connected; and

connecting the conductive shield of the one end of coaxial cable to the first electrode such that the conductive shield and the first electrode are electrically connected.

In another aspect of the second embodiment, the step of providing the second electrode includes the steps of providing a tip stem portion extending from the convex portion, the tip stem portion and the convex portion being electrically connected; and placing a borehole in the tip stem portion.

In another aspect of the second embodiment, the step of the step of connecting the inner conductive core of the one end of coaxial cable to the second electrode includes inserting the inner conductive core of the one end of the coaxial cable into the borehole in the tip stem portion of the second electrode; and connecting the inner conductive core within the borehole to the tip stem portion of the second electrode.

In another aspect of the second embodiment, the method of making a probe includes the step of connecting an electrical circuit to the other end of the coaxial cable, and the electrical circuit configured to receive, via the coaxial cable, electrical signals from the first and second electrodes.

Another aspect of the second embodiment is a method of making a probe including the steps of connecting a probe housing to the second electrode; placing the coaxial cable in the probe housing with a distal end of the coaxial cable extending outside the probe housing; and connecting the distal end of the coaxial cable to an electrical circuit located outside the probe housing.

Another aspect of the second embodiment is a method of making a probe including the steps of providing a tubular connector with a connector central opening and a connector external surface; threading the coaxial cable through the connector central opening; and inserting the tubular connector into the first electrode central opening such that the tubular connector is connected to the first electrode.

In another aspect of the second embodiment, the step of providing a coaxial cable includes the steps of exposing a longitudinal portion of the inner conductive core; and exposing a longitudinal portion of the conductive shield.

A third embodiment of the invention, according to this disclosure, is a method of measuring electrical properties of soil or medium. The method including the steps of providing a probe tip including a first electrode connected to a second electrode including a convex portion, the probe tip including an electrode insulator disposed between the first and second electrode and a coaxial cable with one end connected to the first and second electrodes and another end connected to an electrical circuit; inserting a probe tip into the soil or medium; and transmitting electrical signals from the probe tip, via the coaxial cable, to the electrical circuit.

In another aspect of the third embodiment, the method includes the step of using the probe tip to measure the soil dielectric permittivity.

In another aspect of the third embodiment, the method includes the step of transmitting electrical signals from the probe tip, via the coaxial cable, to the electrical circuit as the probe tip is inserted into the soil or medium; and using the transmitted electrical signals to measure electrical properties of the soil or medium.

In another aspect of the third embodiment, the step of providing a probe tip further includes the step of providing a probe tip with the second electrode connected to a probe housing and the coaxial cable disposed within the probe housing and the distal end of the coaxial cable extending outside of the probe housing, wherein the electrical circuit is connected to the distal end of the coaxial cable external to the probe housing.

In another aspect of the third embodiment, the step of transmitting electrical signals further includes the step of transmitting electrical signals from the probe tip, via the coaxial cable, to the electrical circuit external the probe housing.

### BRIEF DESCRIPTION OF THE SEVERAL VIEWS OF THE DRAWINGS

The foregoing summary, as well as the detailed description of the preferred embodiments of the invention, will be better understood when read in conjunction with the appended drawings. For the purpose of illustrating the invention, there is shown in the drawings, which are diagrammatic, embodiments that are presently preferred. It should be understood, however, that the present invention is not limited to the precise arrangements and instrumentalities shown. In the drawings:

FIG. 1 is a cross sectional view of the probe conical tip electrodes according to one embodiment of this disclosure;

FIG. 2 is a cut away view of the probe conical tip electrodes as shown in FIG. 1;

FIG. 3 is an embodiment, according to this disclosure, of a penetrometer system incorporating and NIR sensor and the probe conical tip electrodes of FIG. 1;

FIG. 4 is a top view of the cross section, along line B-B, of the embodiment of the probe conical tip depicted in FIG. 1;

FIG. 5 is a side cross-sectional view of an embodiment according this disclosure of the conical probe tip of FIG. 1 attached to a sensor body of a probe or penetrometer;

FIG. 6 is flow diagram of an embodiment according to this disclosure of a method of measuring electrical properties of soil using the conical probe tip depicted in FIG. 1; and

FIG. 7 is a flow diagram of an embodiment, according to this disclosure of a method of making the penetrometer system of FIG. 3 to determine the moisture percentage of soil.

### DETAILED DESCRIPTION OF THE INVENTION

Certain terminology is used in the following description for convenience only and is not limiting. The words “inner”, “inwardly” and “outer”, “outwardly” refer to directions toward and away from, respectively, a designated centerline or a geometric center of an element being described, the particular meaning being readily apparent from the context of the description. Also, as used herein, the words “connected” or “coupled” are each intended to include integrally formed members, direct connections between two distinct members without any other members interposed therebetween and indirect connections between members in which one or more other members are interposed therebetween. The terminology includes the words specifically mentioned above, derivatives thereof, and words of similar import.

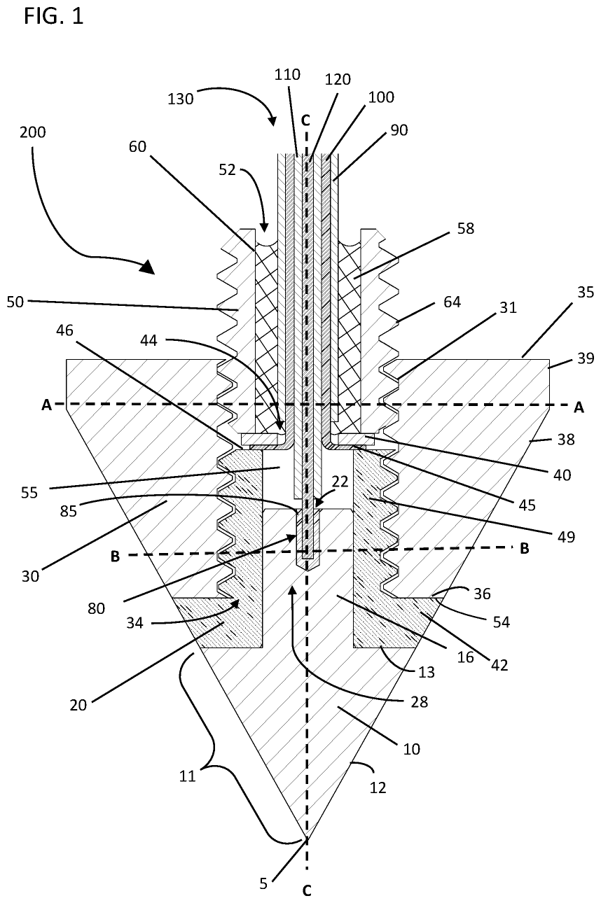

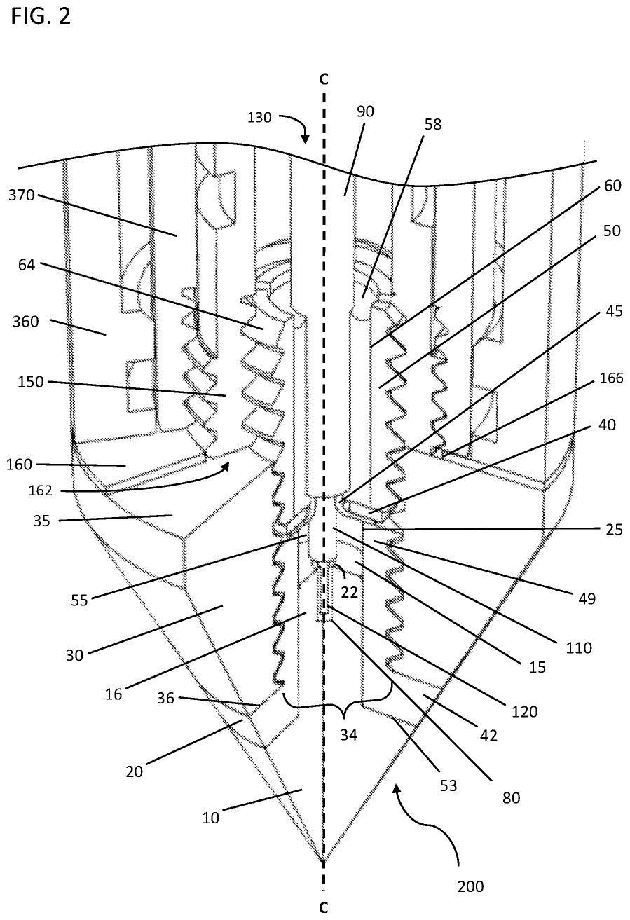

Like numbers are used to indicate like elements throughout. Elements, components, and/or features that are discussed herein with reference to one or more of FIGS. 1-8 may be included in and/or utilized with any of FIGS. 1-8 without departing from the scope of the present disclosure.

The present disclosure discusses the use of a probe tip 200 with respect to measuring the resistance, impedance, dielectric constant and soil water content of soil. Additionally, it is recognized that the penetrometer tip 200 may be used to sense properties and constituents of other media into which it is inserted. Examples of other applicable media include stored grain and other agricultural products, wood chips and other energy producing biomass, and raw materials such as powders etc.

A significant advantage of the penetrometer probe tip 200 of present disclosure over prior physical configurations of penetrometer probe mounted electrodes is that the tip electrodes 10, 30 contact the soil or other media at the face of a convex penetrometer apex tip 5. This allows the probe tip 200 to begin to measure electrical properties of the soil before the full compression and axial displacement the soil will eventually be subjected to as a result of the penetration process. Thus, the probe tip 200 enables measurement of soil electrical properties in situ under greatly reduced disruption of the natural soil in comparison to prior physical configurations.

A further advantage is that electrical signals between the active sensing circuit and/or circuitry 400 and medium under test are carried by a coaxial cable 130 and electrodes 10, 30 that may be coaxial and radially symmetric over the entire signal path between the circuitry 400 and medium under test. The use of the coaxial cable 130 greatly increases immunity to noise and signal corruption from external electromagnetic fields which may include uncontrolled ambient fields, fields produced by circuitry associated with other sensors in the probe or penetrometer 700, self-emitted and internally reflected fields, and other fields.

As shown in FIGS. 1-5, an embodiment of a probe tip 200, according to this disclosure, comprises a first electrode 30, an electrode insulator 20, second electrode 10, and a coaxial cable 130. The probe tip 200 may be utilized on one end of a penetrometer 700 and configured to measure the impedance or other electrical properties of soil or other media in which it is placed. The impedance may then be used to determine other soil or media properties such as the dielectric of the soil or media and soil water content.

Soil is a medium including a three-component mixture of materials in solid, gaseous, and liquid phases. The solid phase consists of soil particles which pack to form a structure or matrix having interconnected pore spaces. These pores contain liquid and/or gas (typically air) into and through which water or other liquid may flow or remain stationary. The dielectric constant of air is 1 by definition. The dielectric constant of dry soils is generally in the range of 2.5 to 4.0, and the dielectric constant of water is around 78 at room temperature. The high contrast between the dielectric constant of water and that of the other two materials in a soil matrix allows the moisture content of soils to be inferred from measurement of the dielectric constant of the mixed media.

The first electrode 30 is annular and tubular and includes an interior surface 31, exterior surfaces 32 and 33, a first end surface 36, a second end surface 35, a first or central opening 34. Additionally, the first electrode 30 is formed about a central axis or centerline C. The interior surface 31 defines first or central opening 34 which extends through the first end surface 36 and second end surface 35. In this embodiment, the interior surface 31 is threaded to allow for rotational or threaded connections to the electrode insulator 20.

The exterior surfaces 32 and 33 are annular and define the perimeter or circumference of the first electrode 30. As shown in FIG. 1, exterior surface 32 is sloped radially and axially, with respect to the central axis C, and defines a conical or frustoconical section 38 (as depicted between line A-A of FIG. 1 and the first end surface 36) with the first end surface 36 being the apex. Accordingly, the diameter of the first end surface 36 is less than the diameter of the second end surface 35. External surface 33 is contiguous with external surface 32, and surface 33 defines an annular or cylindrical section 39 (as depicted between line A-A of FIG. 1 and the second end surface 35).

It is understood that the configuration of the first electrode 30 may include varied configurations. For example, the first electrode 30 may be frustoconical, entirely conical or entirely cylindrical. Additionally, other 3-dimensional configurations may be utilized.

The second electrode 10 comprises a conical or convex portion 11 with a tip stem portion 16 extending therefrom, and a cavity 80 within the tip stem 16. Additionally, the second electrode 10 is formed about the centerline or central axis C. Portion 11 includes an external surface 12 which is sloped with respect to the centerline C and defines the convex portion 11 or cone with an apex 5 and base surface 13. The tip stem 16 extends axially from the base surface 13 and away from the apex 5. Also, the tip stem 16 may be centered over the base surface 13 such that the tip stem 16 and base surface 13 are both formed about the central axis C.

It is noted that portion 11 is shown as conical. Additional forms may also be suitable for penetration of a media or soil. Other convex forms that may be suitable include ojive, elliptical, and pyramidal.

The tip stem 16 has a diameter or width which is less than the diameter or width of both the base surface 13 and first opening 34. Additionally, the tip stem 16 has outer surface 14 which is contiguous with the base surface 13. As shown, the outer surface 14 defines an annular, circular and cylindrical tip stem portion 16. Preferably, the tip stem portion 16 is cylindrical, but other forms such as hexagonal and cuboid may be utilized.

A cavity 80 is formed within the tip stem portion 16 with an open end or opening 22 formed in the second electrode end surface 15. The cavity 80 may be a borehole or blind hole extending axially into the tip stem portion 16 towards the convex portion 11.

The first and second electrodes 30 and 10, respectively, are arranged such that the tip stem portion 16 of the second electrode 10 is within the first opening 34. In this configuration, the first and second electrodes 30, 10 are arranged in-line and concentric about central axis C. Additionally, both electrodes 30, 10 are physically connected, but entirely electrically insulated from one another as well as entirely physically separate and spaced apart.

An electrode insulator 20, which is also formed about the central axis or centerline C is placed between the first and second electrodes 30 and 10, respectively. The insulator 20 maintains physical separation of the two electrodes 30, 10 as well as electrical insulation of the two electrodes 30, 10 from one another. As shown in FIGS. 1-2 and 5, the insulator 20 is tubular with an L or T shaped cross section and includes a substantially radially extending annular portion 42, a substantially axially extending annular and tubular portion 49, an interior surface 25 and a central or second opening 28.

Radially extending portion 42 is formed about the central axis C and includes radially extending upper and lower surfaces 54 and 53, respectively. Portion 42 is disposed between and abuts the first and second electrodes 30, 10 such that the electrodes 30, 10 are axially spaced apart. More specifically, the surface 54 is connected to and/or abuts the first electrode first end surface 36, and surface 53 is connected to and/or abuts second electrode base surface 13.

Axially extending portion 49 is formed about the central axis C and may be cylindrical. Portion 49 extends axially from portion 42 into the first opening 34 such that portion 49 provides radial separation between the first and second electrodes 30, 10 extending along central axis C. The exterior surface 41 defines an annular, circular or cylindrical form of portion 49, and the exterior surface 41 is connected to and/or abuts the interior surface 31 of the first electrode 30 and the cylindrical outer surface 14 of the second electrode 10. Additionally, for connection to the first electrode 30, the exterior surface 41 may include threads that correspond to the threads on interior surface 31 of the first electrode 30.

Interior surface 25 defines a second central opening 28 that extends through portions 42 and 49 and may also be formed about the central axis C. The tip stem portion 16 is inserted into opening 28 and the cylindrical outer surface 14 is connected to and/or abuts interior surface 25. The tip stem portion 16 may include a height, or axial length, that is less than the combined height of the insulator 20 and first electrode 30. As a result, the tip stem portion 16 may only advance into a portion of the height or length of the first opening 34 leaving an unfilled section or space 55 in the central opening 34.

Within the first opening 34 and directly above the tip stem portion 16, the perimeter of the space 55 may be defined by the insulator interior surface 25. Axially, the space 55 may extend from the insulator upper surface 46 to the top of the tip stem portion 16 which is the second electrode end surface 15. If preferred, the space 55 may be filled with a suitable insulating material such as a non-conductive epoxy potting compound.

The connection of the first and second electrodes 30, 10 and the insulator 20 may be a combination of a threaded and press fit connections. The exterior surface 41 of the insulator 20 may be threaded in a manner corresponding to the threads on the interior surface 31 of the first electrode 30. Using the threads, the insulator 20 may be rotationally inserted into the first electrode 30. Then, tip stem portion 16 may be inserted into central opening 28 of the insulator 20 and the second electrode 10 connected to the insulator using a press fit connection between the tip stem portion 16 and the interior surface 25. Additionally, other types of connections are contemplated. For example, the press fit connection may also include threads and either the threaded or press fit connection may also utilize suitable bonding agents such as adhesives and epoxies.

A coaxial cable 130 is placed within the central opening 34, space 55 and cavity 80. Preferably, the coaxial cable 130 is arranged along the central axis C such that it may be coaxially and/or concentrically or approximately concentrically arranged with the first electrode 30, insulator 20 and second electrode 10. The coaxial cable 130 comprises concentrically arranged layers or elements including: an inner conductive core 120, a core insulator 110, a conductive shield 100, and an outer insulator 90. The most central element is the inner conductive core 120 which is circumferentially surrounded by the core insulator 110 which is circumferentially surrounded by a conductive shield 100 and then, the outer insulator 90 surrounds the entire shield 100. Preferably, the inner conductive core 120 and the conductive shield 100 may be formed of copper, but as is known to one of ordinary skill in the art, other suitable conductive materials such as metals, metallic compounds and/or metal alloys may be utilized. Additionally, the outer insulator 90 and core insulator 110 may be formed of insulating materials known to one of ordinary skill in the art.

The conductive core 120 is physically and electrically connect to the second electrode 10 but insulated from the first electrode 30. Preferably, this is accomplished by inserting the core 120 free from the other layers of the coaxial cable 130 into the cavity 80. An electrically conductive bonding agent 85 such as a silver filled epoxy and/or adhesive may be utilized to secure the core 120 to the second electrode 10 while maintaining the electrical connection. Other suitable methods of physically and electrically connecting the core 120 to the second electrode 10 may also be utilized including conductive soldering and other conductive bonding agents including adhesives and epoxies, etc.

Within the space 55, the core 120 is covered by the core insulator 110. This allows the core 120 to remain insulated from the shield 100 which may be exposed above the insulator upper surface 46.

The conductive shield 100 is electrically and physically connected to the first electrode 30 but insulated from the second electrode 10. This may be accomplished within the central opening 34 by bending the conductive shield 100 such that it has a radially extending portion 101. Next, portion 101 is positioned such that it abuts a radially extending electrically conductive member 40 and the insulator upper surface 59.

As shown in FIG. 1, member 40 extends axially between conductive shield 110 and tubular connector 50 as well as potting material 58 if present. Member 40 may be an annular or circular disc with a third or tubular connector central opening 44. For insertion into the opening 34, member 40 may have an outer diameter that is relatively smaller than the diameter of opening 34. Also, the center opening 44 should be large enough for insertion of the conductive shield 100. On example of a suitable conductive member may be a copper washer, but it is envisioned that other suitable metals, metallic components and metallic alloys may be used. Additionally, the shape of member 40 may also be varied.

Member 40 allows the conductive shield 100 to form electrical continuity with the first electrode 30 via conduction through the connector 50. The shield 100 may be directly connected to member 40 and held in place by compression between the connector 50 and the insulator 20 and/or a soldering or a conductive bonding agent may be utilized. Additionally, the member 40 may also be configured to provide direct contact with the interior surface 31 of the first electrode 30.

The portion of the coaxial cable 130 above member 40 is covered with the outer insulator 90. More specifically, the coaxial cable 130 is covered by insulator 90 from conductive member 40 through the length of the probe and to the surface. At the surface, the cable 130 may connect to electronic circuitry 400 and/or a computer system(s) 500 for data collection, interpretation and control of the circuitry.

The cable 130 and connection to the probe tip 200 is axially aligned and concentric with the probe tip 200, such that the conductive paths formed by the tip 200 and cable 130 are coaxially arranged over the entire path between electronic circuitry 400 and contact with the media under test. This provides a continuously coaxial signal path for electrical signals conducted between the electronic circuitry 400 and the media. Providing a path for electrical signal conduction that is coaxially arranged is beneficial because the cable 130 is highly immune to electrical noise and interference over its entire length from the soil contact interface of the electrodes 10, 30 to the electronic circuitry 400. Also, the electronic circuitry 400 may be located outside the probe 700 thereby leaving more space available inside the probe for additional sensors and circuitry such as optical sensors, acoustic sensors, force sensors, and other sensors.

It will be understood by a practitioner of ordinary skill in the art that the shapes, dimensions, and geometric proportions of the parts and components forming the electrical signal path through the probe conical tip electrodes, in this embodiment the first and second electrodes 30, 10, insulator 20, tip stem portion 16, member 40, space 55, their inner, outer, upper and lower surfaces may be varied to assure that the electrical signal path through the coaxial cable 130 and connected parts is of substantially uniform and minimally varying electrical impedance over its entire extent from electronic circuitry 400 to contact with the soil.

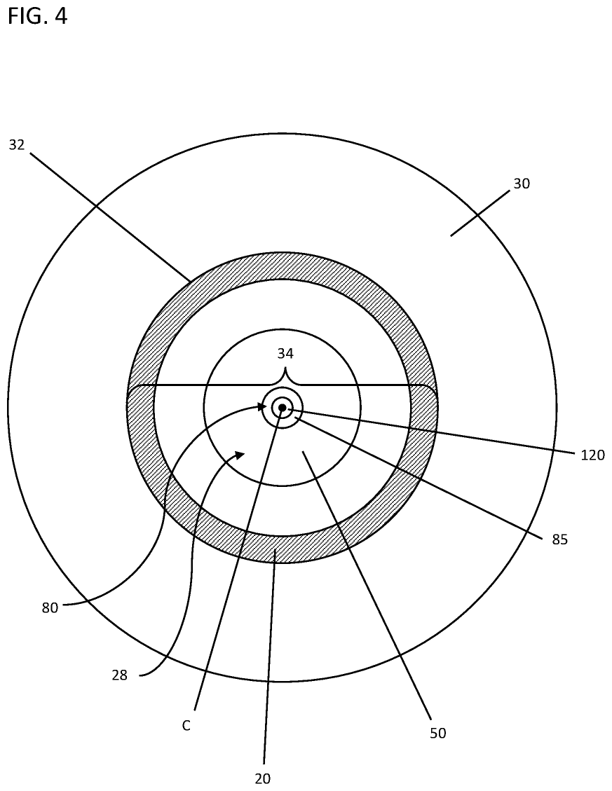

FIG. 4 depicts a top view of the cross section of the probe tip 200 along line BB of FIG. 1. As shown, the conductive core 120, first electrode 30, insulator 20 and second electrode 10 may be coaxial and concentric about the centerline C.

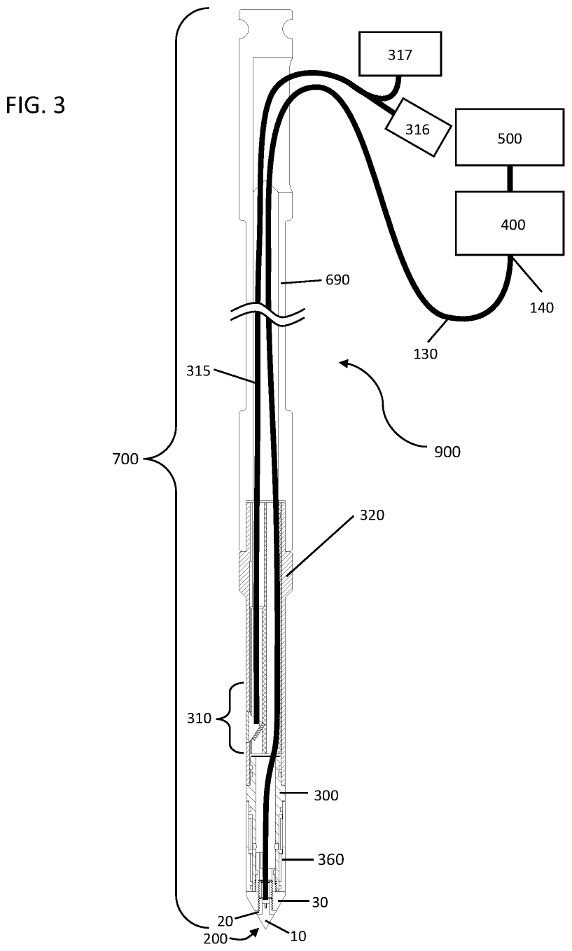

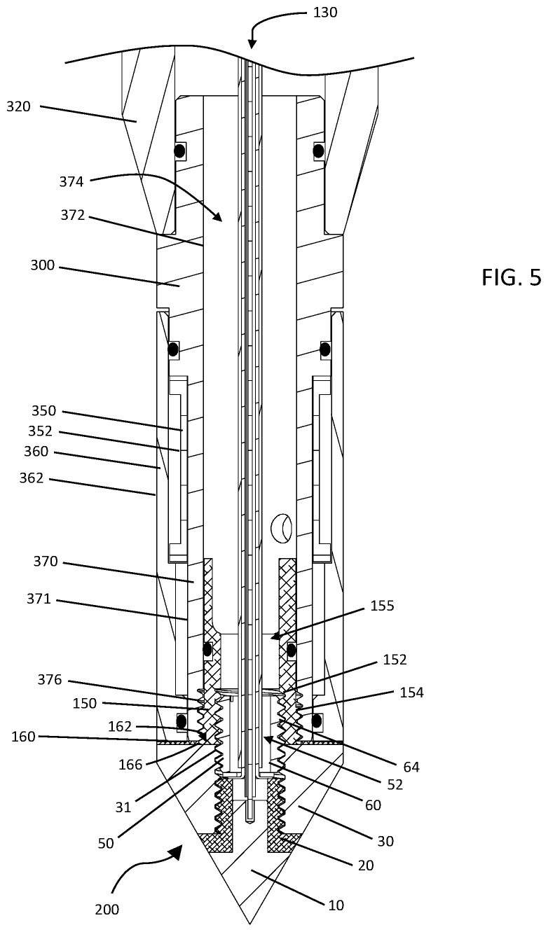

The probe tip 200 may be directly connected to a push rod, as shown in FIGS. 3 and 5, more preferably connected to a sensor body 300 which may contain, as shown in FIG. 3, additional sensors and is further connected to a probe housing 320 which may include, yet, more sensors such as the near-infrared reflectance (NIR) sensor that comprises a downhole NIR sensor assembly 310, optical fiber 315, up-hole light source 316 and NIR spectrometer 317 as disclosed in U.S. application Ser. No. 17/187,833 which is herein incorporated by reference in its entirety. The device configuration provides a coaxial arrangement of electrodes 10 and 30 to which connects a coaxial cable 130 leading to electronic test circuitry 400 and computer system 500.

FIG. 5 is a cross-sectional view of an embodiment according to this disclosure of the probe tip 200 connected to a sensor body 300. To sense soil mechanical properties the sensor body 300 may incorporate a sleeve load cell sensor 350 positioned between a friction sleeve 360 and tip mandrel 370 and having an outer surface 362 in contact with the soil. The sleeve load cell sensor 350 has an outer surface 352 to which is affixed one or more strain gages (not shown) to measure load on the sleeve load cell sensor 350 generated by friction exerted on the outer surface 362 of the friction sleeve 360 by soil outside the probe. The mandrel 370 is tubular and has a mandrel outer surface 371 to which may be affixed one or more strain gages not shown situated below the sleeve load cell 350 to measure the axial load transmitted to the mandrel 370 from the probe tip 200. The mandrel 370 has a mandrel interior surface 372 which defines a central opening 374 through which cable 130 travels from the probe tip 200 to the surface and/or electrical circuit 400 and computer system 500. The interior surface 372 also may incorporate threads 376 to assist in the connection of the probe tip 200 to the sensor body 300.

The friction sleeve 360 may be made of a material that remains 3-dimensionally stable under the forces that a penetrometer or probe may face from the media in which the penetrometer or probe is inserted. Examples of suitable materials include stainless steel, titanium, other metals, metallic compounds, etc.

A pair of reciprocal tubular threaded connectors 50, 150 may be utilized to connect to the probe tip 200 to the sensor body 300. As shown in FIGS. 1-2 and 5, connector 50 is tubular with an exterior threaded surface 64 and an interior surface 60 which defines a connector central opening 52. The threaded surface 64 corresponds to the first electrode interior threaded surface 31. Connector 50 may be rotationally advanced into central opening 34 until the connector 50 abuts the electrically conductive member 40.

The coaxial cable 130 is disposed within the central opening 52. As shown in FIG. 1, an insulating potting material 58 may be placed between the interior surface 60 and the cable 130 outer insulator 90. The potting material 58 may assist in securing the cable 130 in place and preventing contaminants such as water, moisture or particles from entering the first electrode central opening 34. Examples of suitable potting materials may include epoxy potting compounds, thermoset polymers, injection molded thermoplastics, etc.

Connector 150 is also tubular and may include both internal and external surfaces 152, 154, respectively, which in the described embodiment are threaded but may be smooth or have other surface characteristics suitable for connections. The internal surface 152 defines a central opening 155 which has diameter and threading suitable for receiving the corresponding threaded exterior surface 64 of connector 50. Coaxial cable 130 is disposed within the central opening 155 of connector 150. Although not shown, to secure the cable 130, potting material 58 may also be disposed between the interior surface 152 and the outer cable insulator 90.

In the preferred embodiment connectors 50, 150 are formed of an electrically non-conductive material(s) of suitable strength to secure the probe tib 200 to the sensor body 300 and withstand the stresses which occur during insertion of the probe tip 200 into soil or other media. Examples of suitable materials include sufficiently hard plastics, rubber and ceramics, or combination thereof, etc.

An electrically insulating element 160, extends substantially radially, with respect to the central axis C and entirely surrounds connector 150. The insulating element 160 may be annular with an insulating element central opening 162 defined by an interior surface 166 which completely surrounds the entire exterior surface 154 of connector 150. The lower surface of the insulating element 160 abuts the first electrode 30 upper surface 35, and the upper surface of element 160 that abuts the friction sleeve 360 and tip mandrel 370 such that there is no electrical contact between the tip mandrel 370 and the first electrode 30, nor between the friction sleeve 360 and the first electrode 30.

The insulating element 160 may provide environmental seal to prevent elements such as moisture, particles, water, etc. from entering the central opening 34 and contacting the conductive shield 100, conductive core 120 and or conductive member 40. Additionally, the insulating element 160 provides electrical insulation to electrically isolate the friction sleeve 360, tip mandrel 370 and sleeve load cell 350 from the probe tip 200. To accomplish both of these functions, the insulating element 160 may abut the upper surface 35 of the first electrode 30, connector 150, tip mandrel 370 and friction sensor sleeve 360 around the entire circumference of the connector 150 and first electrode 30.

The insulating element 160 may comprise a strong, nonpliable insulating material that has a very low permeability to water and other liquids, so its electrical conductivity and dielectric permittivity will remain stable when exposed to liquids. Suitable materials may include, for example, plastics, resins, ceramics and other compositions such as a 20% glass fiber reinforced (i.e., “glass-filled”) Delrin (acetal resin), glass-filled PEEK (PolyEthylEther-Ketone), ceramics and other composite materials such as fiber glass (i.e., FR-4).

The insulating element 160 may be secured in place by the force of the connection between the probe tip 200 and sensor body 300. Additionally, a suitable bonding agent including adhesives and epoxies, etc. may also be utilized.

The probe tip 200 may be used as part of a penetrometer system 900 to perform various studies of soil or other media into which it is inserted. Some examples of the different properties which may be studied include soil moisture, dielectric permittivity, complex dielectric, electrical conductivity and resistivity, electrical impedance spectroscopy (EIS) for obtaining information related to chemical species present rather than a measurement of a direct electrical property of the soil, linear sweep voltammetry (LSV), and eH—a measure of the redox (oxidation-reduction) state of a solution, and other measurements of electrical response of the soil, etc. Additionally, a computer system or handheld device 500 including graphical user interface (GUI) may be connected to the circuitry 400 and utilized to display the measurements performed by the penetrometer system 900.

To perform the studies, the penetrometer system 900 includes the probe tip 200, the circuitry 400, and the coaxial cable 130 connected therebetween. The measurements may include dielectric constant and resistivity, etc. One end of the coaxial cable 130 is connected to the probe tip 200 and the other end is connected to the circuitry 400. The use of the coaxial cable 130 reduces variables in capacitance of the system. The circuitry 400 may include an oscillator, resistivity and analog switching circuit that may use the probe tip 200 to take electrical measurements such as dielectric permittivity and resistivity.

The two tip electrodes 10 and 30 are separated by an insulator 20 that maintains the constant spacing between electrodes 10, 30. This design ensures that capacitance of the tip 200 remains constant except as affected by the soil through which the electrical field may also flow. Once the tip 200 is fully implanted into the soil the area surrounding the tip 200 will add parallel capacitance to the fixed capacitance of the tip. This surrounding area extends 360 degrees around the probe tip 200 and may be referred to as the area of influence. The dielectric permittivity of the area of influence may range from one in air to about eighty in water. For soils, being a mixture of soil solids, air, and water, the dielectric permittivity may range from about two-and-a-half to about thirty-five.

The soil moisture circuit 400 may be an LC electronic oscillator such as a modified Clapp oscillator which oscillates around 70 MHz when the tip 200 is surrounded by air. The capacitance (C) is a series-parallel combination of the in-circuit capacitors including the coaxial cable 130, tip 200, and the area of influence in the soil. The inductor is a series combination of the in-circuit inductors, circuit traces, coaxial cable 130 and the tip electrodes 10, 30. The Clapp oscillator is very stable with slight changes in the value of the inductors. A practitioner of ordinary skill in the art will recognize that carefully selection the fixed values of discrete inductive (L) and capacitive (C) elements in the circuit, the oscillator will allow the tip 200 capacitance determined by the soil dielectric permittivity to dominate the resonate frequency. By measuring changes in frequency of oscillation, the capacitance at the tip 200 and therefore the soil dielectric permittivity due to the area of influence may thereby be determined.

Topp's equation, as is known by one of ordinary skill in the art, may be used to predict the percent volumetric moisture (Vm) in soil based on its dielectric permittivity, as can Ledieu's equation, Ferre's equation, or any soil-specific equation relating soil moisture content to soil dielectric permittivity. The penetrometer 700 and/or penetrometer system 900 may be utilized to determine the dielectric constant of the unknown soil, and the determined dielectric constant may be utilized to determine the percent Vm, based on an appropriate equation.

In addition to the using an oscillating electronic circuit to measure dielectric permittivity, a variety of electrical circuit types can incorporate electrodes 10, 30 to effect measurement of additional soil electrical characteristics such as electrical resistivity, conductivity, spectral impedance, and other electrical characteristics.

An example of the use of the penetrometer system 900, according to this disclosure, is to determine and monitor the percent Vm that is in the soil of interest as it pertains to crop health. This may be performed by collecting soil electrical data which corresponds to soil characteristics and performing the necessary calculations. It is known by one of ordinary skill in the art that the dielectric constant of a soil-water matrix increases as the percent of water is increased. Also, Topp's equation, as is known by one of ordinary skill in the art, may be used to predict the percent volumetric moisture (Vm) in soil based on its relative dielectric constant. Therefore, the penetrometer system 900 may be utilized to determine the dielectric constant of the unknown soil, and the determined dielectric constant may be utilized to determine the percent Vm, based on Topp's equation.

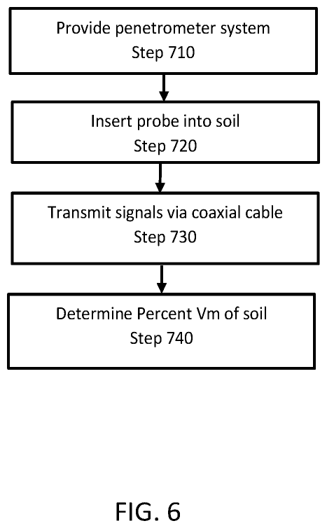

A method of using the penetrometer system 900, according to this disclosure, to make electrical measurements of soil is depicted in FIG. 6. Initially, in Step 710, the penetrometer system 900 including the probe tip 200, coaxial cable 130, circuitry 400, and computer system 500 as described above is provided. Next, in step 720, the probe tip 200 is inserted into the soil. As the probe tip 200 is advanced into the soil, in step 730, electrical measurements of the soil at the tip of a penetrometer are performed by interaction of the electrical circuitry 400 with the soils via transmission of electrical signals between the circuitry 400 and the soils through the coaxial cable 130 and electrodes 10, 30. In other words, the signals are transmitted along a signal path comprising conductors 10, 30, 100, 120 arranged coaxially over their entire length. This allows electrical measurements to begin upon insertion of the probe tip 200 into the soil, or the measurements may be made in-situ at a desired depth. As discussed above, a principal advantage of the penetrometer system 700 is that the measurements may be made with the soil in a virtually undisturbed state.

In step 740, the computer system 500 may receive the electrical signal data from the circuitry 400. Next, the computer system 500 may utilized to the electrical data to determine the dielectric constant of the soils and then, determine the soil percent Vm using the dielectric constant and Topp's equation.

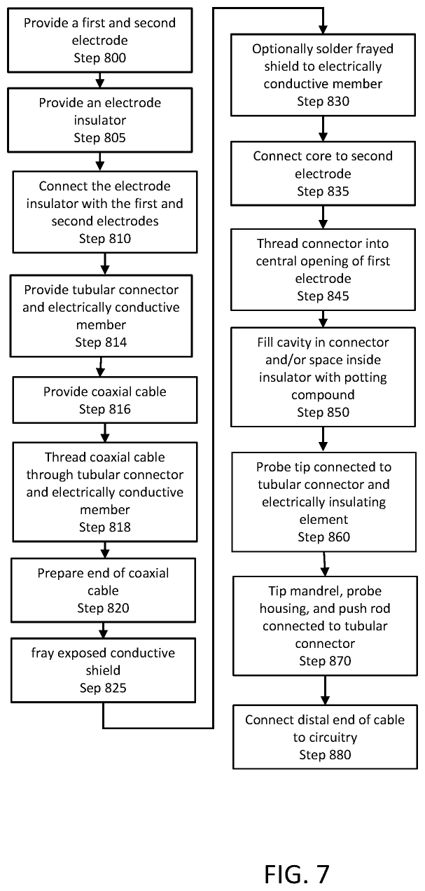

FIG. 7 is a flow diagram of an embodiment, according to this disclosure, of a method of making the penetrometer system 900. Initially, in step 800, the first electrode 30 and second electrode 10 are provided according to the above description. The first and second electrodes 30, 10 preferably comprise corrosion resistant, highly conductive materials with suitable strength for penetration of the desired media. For example, the electrodes may be formed of metals, metallic compounds and/or metal alloys including 308 stainless steel, 316 stainless steel, 17-4 stainless steel and/or, grade 5 titanium (Ti 6A-4V), copper compounds, nickel etc.

Both electrodes 30, 10 may comprise the same materials such as stainless steel. Alternatively, the first and second electrodes 30, 10 may be made of different materials such as two different stainless steel compounds or one stainless steel compound and a copper compound.

Techniques such as casting, molding as well as CNC machining and other types of milling and additive manufacturing may be utilized to achieve the desired shape and configuration.

In step 805, an electrode insulator 20, as discussed above, is provided. The insulator 20 may comprise a strong, nonpliable insulating material that has a very low permeability to water and other liquids, so its electrical conductivity and dielectric permittivity will remain stable when exposed to liquids. Suitable materials may include, for example, fiber glass, resins, ceramics and other compositions such as a 20% glass fiber reinforced (i.e., “glass-filled”) Delrin (acetal resin), glass-filled PEEK (PolyEtherEther-Ketone), ceramics and other composite materials such as fiber glass (i.e., FR-4).

Preferably, insulator 20 may be formed with portions 42 and 49 being integral. It is also envisioned that portions 42 and 49 may be formed separately and bonded together with a suitable adhesive or epoxy, etc.

The insulator 20 may be fabricated using processes known to one of ordinary skill in the art including injection molding, additive manufacturing, etc.

In step 810, the electrode insulator 20 may be connected between the first electrode 30 and second electrode 10 such that the first electrode 30 is axially and radially spaced apart from the second electrode 10. The axially extending portion 49 of the insulator 20 may be rotationally advanced in the central opening 34 of the first electrode 30 until the radially extending portion 42 of insulator 20 abuts surface 36 of electrode 30. The tip stem portion 16 of the second electrode 10 may be inserted into the insulator 20, and a press fit connection may be made between the second electrode 10 and insulator 20. Additionally, insulator 20 may be secured to the first electrode 30 utilizing a suitable bonding agent such as an adhesive glue or epoxy.

In step 814, the tubular connector 50 and electrically conductive member 40, as described above, may be provided.

In step 816, a coaxial cable 130 of suitable length to extend from the probe tip 200, through the penetrometer 700, to the above ground electrical circuitry 400 is provided. Some non-limiting examples of suitable cables include RG-50, RG-174, RG-50 NU and other standard and non-standard coaxial cables.

Next, in step 818, one end of the coaxial cable 130 may be threaded through the tubular connector 50 central opening 44 and the conductive member 40.

In step 820, one end of the coaxial cable 130 is prepared for attachment to the first electrode 30 and second electrode 10. As shown in FIGS. 1, 2 and 5, the inner conductive core 120 may be exposed by removing an appropriate length of the core insulator 110, conductive shield 100, and outer insulator 90. At a point further from the end of the coaxial cable 130 and at a point corresponding to the position of attachment to the first electrode 30, the outer insulator 90 is removed to expose a portion of the conductive shield 100. The length of exposed conductive shield 100 should correspond to the distance required to connect the conductive shield to the electrically conductive member 40.

In step 825, the exposed portion 45 of conductive shield 100 is frayed or pulled outwardly from the insulator 110 such that it is at an angle relative to the centerline C.

In step 830, the exposed portion 45 of the conductive shield 100 is optionally connected to the conductive member 40. If desired, this may be done, for example, by soldering or using an electrically conductive bonding agent.

In step 835, the exposed core 120 is physically and electrically connected to the second electrode 10. An electrically conductive bonding agent 85 may be disposed within the cavity 80 and/or on the exposed core 120. Next, the exposed core 120 may be inserted into the cavity 80. The electrically conductive bonding agent 85 may be suitable adhesives and epoxies such as, such as silver epoxy, graphite filled silicone adhesive, silver-filled polyurethane adhesive, metal-filled snap-cure frozen epoxy, electrically conductive adhesive.

Once the bonding agent 85 has cured, in step 845, tubular connecter 50 may be rotationally advanced into central opening 34 of the first electrode 30. The connector 50 may be advanced within the central opening until the connector 50 and conductive member 40 abut and the exposed portion 45 is pressed between the conductive member 40 and insulator 20.

In step 850, the central opening 52 of tubular connector 50 may be filled with a potting material 58. Optionally, a potting material 58 may also be disposed in space 55 by flowing through openings in the frayed exposed portion 45 of conductive shield 100. As is known by one of ordinary skill in the art, heat may be applied to aid in the curing of the potting material 58.

In step 860, the tubular connector 150 and insulating element 160, as described above, may be provided and connected to the probe tip 200. The other or distal end 140 of the coaxial cable 130 is inserted through tubular connecter 150 and opening 162 of insulating element 160. Tubular connectors 50 and 150 are rotationally advanced or screwed together. Also, the insulating element 160 is placed on the first electrode 30 and around connector 150 such that the insulating element 160 is in direct contact with the second end surface 35 of the first electrode 30.

In step 870, a tip mandrel 370, probe housing 320 and push rod 690, as described above, may be provided and connected to the probe tip 200 via connector 150 and insulating element 160. The distal end 140 of the coaxial cable 130 may be threaded through tip mandrel 370, probe housing 320 and push rod 690. The tip mandrel 370 is rotational advanced or screwed on to connector 150 until it abuts the insulating element 160. The probe housing 320 may be connected to the tip mandrel 370 via a press fit connection or other connections known by one of ordinary skill in the art. The push rod 690 may be connected to the probe housing 320 via set screws not shown extending radially through push rod 690 or by way of a threaded connection or other methods known by one of ordinary skill in the art.

In step 880, the other end 140 of the coaxial cable may be connected to the circuitry 400, which is described above, and the circuitry may be attached to computer system 500. The connection the other end 140 of the coaxial cable 130 as well as the circuitry to the computer system 500 may be made by field terminated SMA connector, BNC connector, or other methods known by one of ordinary skill in the art.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as generally defined in the appended claims.

1. A probe for penetrating and measuring electrical properties of a soil, the probe comprising:
a tubular first electrode including an interior surface defining a central opening extending through the first electrode;
a second electrode including a convex section extending away from the first electrode, the convex section configured for insertion into soil;
an electrode insulator between the first and second electrodes; and
a coaxial cable within the central opening of the first electrode, the coaxial cable including an inner conductive core surrounded by a conductive shield with a core insulator therebetween, the inner conductive core of one end of the coaxial cable connected to the second electrode, and the conductive shield of the one end of the coaxial cable connected to the first electrode.

2. The probe of claim 1, further comprising:
a sensor body connected to the first electrode; and
a probe tip insulator disposed between the sensor body and the first electrode, the probe tip insulator extending substantially radially.

3. The probe of claim 1, further comprising:
an electrical circuit connected to a second end of the coaxial cable, the electrical circuit configured to transmit, via the coaxial cable, electrical signals to the first and second electrodes.

4. The probe of claim 3, wherein the electrical circuit further comprises a Clapp oscillator.

5. The probe of claim 3, further comprising:
a probe housing connected to the second electrode and the coaxial cable extending from the first and second electrode through the probe housing; and
a distal end of the coaxial cable extending outside the probe housing, the distal end of the coaxial cable connected to the electrical circuit outside the probe housing.

6. The probe of claim 1, wherein the tubular first electrode further comprises:
a first end surface and a second end surface with the interior surface extending within the tubular electrode from the first end surface to the second end surface, and the central opening is defined by the interior surface extending through the first electrode, first end surface and the second end surface.

7. The probe of claim 1, wherein the second electrode further comprises:
a third end surface opposite the convex portion; and
a tip stem portion on the third end surface, the tip stem portion extending away from the convex portion, the tip stem portion having a smaller width than the third end surface and the tip stem portion positioned within the central opening of the first electrode.

8. The probe of claim 7, wherein the electrode insulator extends into the central opening of the first electrode and the electrode insulator is between the tip stem portion of the second electrode and the interior surface of the first electrode.

9. The probe of claim 7, further comprising:
a cavity within the tip stem portion of the second electrode, the cavity configured to receive the conductive core of the coaxial cable.

10. The probe of claim 1, wherein the convex portion is solid.

11. The probe of claim 1, wherein the convex portion includes a conical exterior surface.

12. The probe of claim 8, further comprising:
a conductive member disposed on the electrode insulator within the central opening, the conductive member connected to the first electrode and the conductive shield of the coaxial cable.

13. The probe of claim 12, wherein the conductive member is an annular disc with a hollow center, and the inner conductive core and cable insulator are within the hollow center.

14. The probe of claim 1, wherein the first electrode, second electrode and insulator are concentrically connected.

15. The probe of claim 1, wherein the first electrode and second electrode are comprised of a metal or a metallic compound.

16. The probe of claim 1, wherein the first electrode comprises a first compound and the second electrode comprises a second compound, wherein the first compound and second compound comprise different metals or metallic compounds.

17. A method of making a probe to measure electrical properties of soil or medium, the method comprising the steps of:
providing a tubular first electrode including an interior surface defining a first electrode central opening extending through first electrode;
providing a second electrode including a convex portion configured for insertion into the soil;
providing an electrode insulator configured to be placed between the first and second electrodes and inside the first electrode central opening;
inserting the electrode insulator into to the central opening of the first electrode such that the electrode insulator and first electrode are connected;
connecting the second electrode to the electrode insulator;
providing a coaxial cable with one end including an inner conductive core, a conductive shield, and a core insulator therebetween;
inserting the one end of the coaxial cable through the first electrode central opening;
connecting the inner conductive core of the one end of coaxial cable to the second electrode such that the inner conductive core and the second electrode are electrically connected; and
connecting the conductive shield of the one end of coaxial cable to the first electrode such that the conductive shield and the first electrode are electrically connected.

18. The method of claim 17, wherein the step of providing the second electrode further comprises the steps of:
providing a tip stem portion extending from the convex portion, the tip stem portion and the convex portion being electrically connected; and
placing a borehole in the tip stem portion.

19. The method of claim 17, wherein the step of connecting the inner conductive core of the one end of coaxial cable to the second electrode, further comprises the steps of:
inserting the inner conductive core of the one end of the coaxial cable into the borehole in the tip stem portion of the second electrode; and
connecting the inner conductive core within the borehole to the tip stem portion of the second electrode.

20. The method of claim 17, further comprising the step of:
connecting an electrical circuit to a distal end of the coaxial cable, the electrical circuit configured to receive, via the coaxial cable, electrical signals from the first and second electrodes.

21. The method of claim 20, further comprising the steps of:
connecting a probe housing to the second electrode;
placing the coaxial cable in the probe housing with a distal end of the coaxial cable extending outside the probe housing; and
connecting the distal end of the coaxial cable to an electrical circuit located outside the probe housing.

22. The method of claim 17, further comprising the steps of:
providing a tubular connector with a connector central opening and a connector external surface;
threading the coaxial cable through the connector central opening; and
inserting the tubular connector into the first electrode central opening such that the tubular connector is connected to the first electrode.

23. The method of claim 17, wherein the step of providing a coaxial cable further comprises the steps of:
exposing a longitudinal portion of the inner conductive core; and
exposing a longitudinal portion of the conductive shield.

24. A method of measuring electrical properties of soil or medium, the method comprising the steps of:
providing a probe tip including a first electrode connected to a second electrode including a convex portion, the probe tip including an electrode insulator disposed between the first and second electrode and a coaxial cable with one end connected to the first and second electrodes and a distal end connected to an electrical circuit;
inserting a probe tip into the soil or medium; and
transmitting electrical signals from the probe tip, via the coaxial cable, to the electrical circuit.

25. The method of claim 24, further comprising the step of:
using the probe tip to measure the soil dielectric permittivity.

26. The method of claim 24, further comprising the step of:
transmitting electrical signals from the probe tip, via the coaxial cable, to the electrical circuit as the probe tip is inserted into the soil or medium; and
using the transmitted electrical signals to measure electrical properties of the soil or medium.

27. The method of claim 24, wherein the step of providing a probe tip further comprises the step of:
providing a probe tip with the second electrode connected to a probe housing and the coaxial cable disposed within the probe housing and the distal end of the coaxial cable extending outside of the probe housing, wherein the electrical circuit is connected to the distal end of the coaxial cable external to the probe housing.

28. The method of claim 27, wherein the step of transmitting electrical signals further comprises the step of:
transmitting electrical signals from the probe tip, via the coaxial cable, to the electrical circuit external the probe housing.
